(19) United States
(12) Patent Application Publication
(10) Pub. No.: US 2015/0224136 A1
(43) Pub. Date: Aug. 13, 2015

(54) Performance-Enhancing Nasal Irrigation

(71) Applicant: Backed-by-Science, Inc., Yardley, PA (US)

(72) Inventors: Thomas Parker, II, Chalfont, PA (US); Robert Tobin, Bryn Mawr, PA (US)

(21) Appl. No.: 14/175,224

(22) Filed: Feb. 7, 2014

(51) Int. Cl. A61K 33/06; A61K 31/198; A61K 31/7004; A61K 31/522; A61K 31/04; A61K 33/14; A61K 31/197

(52) U.S. Cl. CPC A61K 33/06; A61K 33/14; A61K 31/198; A61K 31/197; A61K 31/522; A61K 31/04; A61K 31/7004

(57) ABSTRACT

Novel compositions for performance-enhancing nasal irrigation, nasal irrigation apparatus for delivering said performance-enhancing compositions, and methods for enhancing performance using said compositions. Any combination of salt, baking soda, one or more alkaline earth metals, one or more amino acids, one or more natural antioxidants, one or more plant-based stimulants, one or more vitamins, and one or more synergists or other ingredients are disclosed. The ingredients described herein may be delivered as an anhydrous powder packet containing water-soluble forms of the component ingredients, and may be delivered along with nasal irrigation devices and instructions for mixing the powder with a fixed volume of pure water.

### BACKGROUND OF THE SUBJECT DISCLOSURE

1. Field of the Subject Disclosure

The present subject disclosure relates to performance supplements. In particular, the present subject disclosure relates to an apparatus, composition, and method of use for performance-enhancing nasal irrigation.

2. Background of the Subject Disclosure

Breathing is a vital aspect of life. Particularly for athletes and other performers, the ability to breathe freely is essential for maximizing oxygen absorption and, therefore, performance. Further, deep breathing enables not only improved oxygenation, but also facilitates elimination of unwanted substances from the body such as carbon dioxide, lactic acid, etc.

Nasal rinsing, also known as sinus irrigation, is an ancient and time-tested method for cleaning one's nasal passages and improving breathing ability. Typically, the nasal passages are rinsed with a saline solution having a salinity that is matched to the salinity of the nasal membranes. This minimizes irritation and facilitates a balanced movement of electrolytes. Further, the salt has some antiseptic effects, minimizing bacterial growth and therefore disease. However, salt water is mildly acidic, and the nasal membrane is slightly alkaline, therefore may get irritated with a saline solution. To counter this effect, sodium bicarbonate (commonly known as baking soda) has been added to existing nasal rinse compositions.

However, even these existing compositions are unable to adequately enhance athletic performance, minimize lactic acid buildup, and provide a pleasurable experience for a user seeking to breathe deeply and freely.

### SUMMARY OF THE SUBJECT DISCLOSURE

The subject disclosure solves the above problems by providing novel compositions for performance-enhancing nasal irrigation, nasal irrigation devices for delivering said performance-enhancing compositions, and methods for enhancing performance using said compositions. Novel compositions described herein are composed of several ingredients, including any combination of salt, baking soda, one or more alkaline earth metals, one or more amino acids, one or more natural antioxidants, one or more plant-based stimulants, one or more vitamins, and one or more synergists or other ingredients. The ingredients described herein may be delivered as an anhydrous powder packet containing water-soluble forms of the component ingredients, and may be delivered along with nasal irrigation devices and instructions for mixing the powder with a fixed volume of pure water.

### BRIEF DESCRIPTION OF THE DRAWINGS

FIG. 1 shows a nasal irrigation composition in an anhydrous powder packet, according to an exemplary embodiment of the subject disclosure.

FIGS. 2A and 2B show containers for nasal irrigation, according to exemplary embodiments of the subject disclosure.

FIGS. 3A and 3B show methods for nasal irrigation, according to exemplary embodiments of the subject disclosure.

### DETAILED DESCRIPTION OF THE SUBJECT DISCLOSURE

The subject disclosure presents novel compositions for performance-enhancing nasal irrigation, nasal irrigation devices for delivering said performance-enhancing compositions, and methods for enhancing performance using said compositions. The compositions described herein may be delivered as an anhydrous powder packet containing water-soluble forms of the component ingredients, and may be delivered along with nasal irrigation devices and instructions for mixing the powder with a fixed volume of pure water. Alternatively or in addition, a prepared solution may be provided for one-time use, for instance, immediately prior to an athletic event. Although the following embodiments and compositions are described with reference to athletes, they are not limited to such use, and may be readily employed by anyone, including allergy and asthma sufferers, performers, breath-work practitioners, etc. Moreover, as used herein and throughout this disclosure, the term “nasal irrigation” includes any practice of irrigating or otherwise wetting the sinus passages with the disclosed compositions for any purpose, including but not limited to enhancement of breathing, health, or athletic performance.

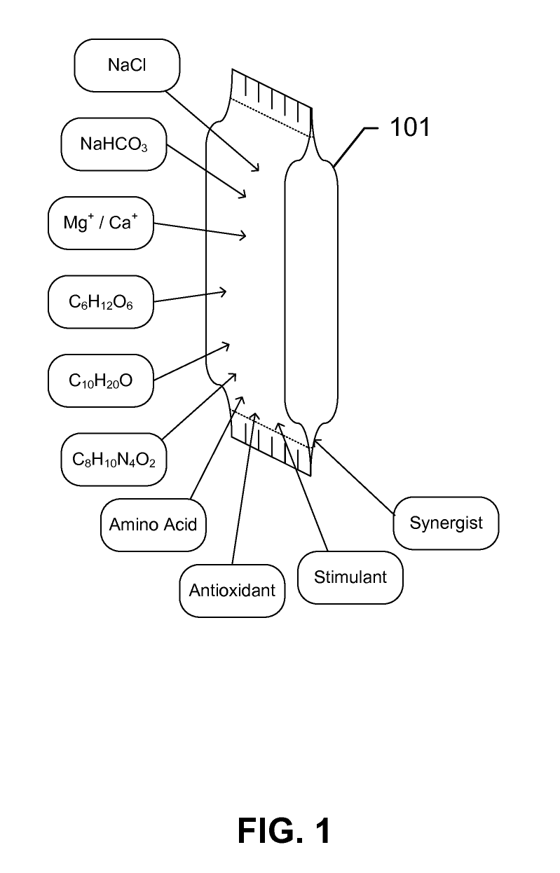

FIG. 1 shows a nasal irrigation composition in an anhydrous powder packet 101, according to an exemplary embodiment of the subject disclosure. Packet 101 may include one or more of the listed ingredients in various proportions, and may be intended to be dissolved in a fixed volume of water, resulting in a solution, emulsion, and/or suspension that is ideal for nasal irrigation. Although several ingredients are listed, the packet may include only some of these, all of these, or additional ingredients not pictured but described in detail herein, in any combination.

For instance, according to an exemplary embodiment of the subject disclosure, packet 101 includes a first percentage of sodium chloride, and a second percentage of sodium bicarbonate. The sodium chloride, commonly known as salt, is intended to match the salinity of the nasal mucosa. Therefore, any amount of salt may be used which, when mixed with water to create an isotonic solution, matches the salinity of the nasal mucosa. The molecular size of salt (around 58.44 g/mol) renders it readily absorbable by nasal membranes. The salt may be of any suitable type identified by the United States Pharmacopeia (USP), or USP-grade.

Solutions of salt, or saline solutions, are typically slightly acidic. Therefore, the addition of sodium bicarbonate, commonly known as baking soda, neutralizes any potential acidity caused by dissolving the salt. Depending on the volume of saline solution, an appropriate amount of baking soda is included in the composition to neutralize the acidity of the saline solution and raise the pH of the solution to a pH similar to that of the nasal mucosa. The resulting solution may be mildly alkaline, similar to the nasal mucosa. Moreover, sodium bicarbonate absorbed into the bloodstream via the nasal mucosa further neutralizes any lactic acid buildup in the blood, thereby increasing athletic performance, particularly under anaerobic conditions, such as for athletes in speed-based events. The sodium bicarbonate has a molar mass of around 84 g/mol, thereby rendering it well-absorbable by the nasal membranes. Further, the sodium bicarbonate may be USP-grade.

In another exemplary embodiment, the composition may include one or more alkaline earth metals in addition to a premeasured amount of salt and baking soda. The one or more alkaline earth metals, such as magnesium or calcium, may be in any water-soluble form, such as magnesium chloride, calcium sulfate, magnesium malate, etc. The one or more alkaline earth metals may be provided in a USP-grade form. Magnesium improves performance by stimulating the synthesis of ATP, therefore providing more energy during muscular exertion. Magnesium has the added advantage of improving lactate metabolism, thereby acting as a buffer for excess lactic acid production. As a result, increased energy and endurance may be experienced by an athlete. Moreover, in quantities of 125 mg or greater per day, magnesium helps stabilize blood sugar and calms the nervous system. Although such a quantity may not be included in the composition, a sufficient quantity may be included to achieve the other positive effects, thereby synergizing and improving the composition. Pure magnesium has a molar mass of 24.3050 g/mol, which is easily absorbed by the nasal membranes. Moreover, magnesium is highly soluble in water, enabling it to be easily added to the solution prior to nasal irrigation. The slightly sour taste of magnesium ions provides a refreshing experience for a user performing the nasal irrigation. In addition, magnesium malate has a molar mass of 156.38 g/mol, which is also easily absorbed by the nasal membranes. The magnesium malate may be USP-grade.

Calcium is an alternative alkaline earth metal that may be used in place of or in addition to magnesium. Calcium's molar mass of 40.078 g/mol enables it to be easily absorbed by nasal membranes and calcium compounds are typically soluble in water.

In exemplary embodiments, one or more essential and non-essential amino acids may be added to the salt and baking soda composition. The amino acids may be added in addition to the alkaline earth metals described above, and may be provided in a USP-grade form. The following amino acids may be added by themselves or in any combination, resulting in a variety of compositions. For instance, beta Alanine is a naturally-occurring non-essential beta amino acid that is a precursor to carnosine, which has a number of beneficial properties, particularly in muscle tissue. Supplementation of the nasal irrigation with a water-soluble form of beta-Alanine may therefore potentially increase the concentration of carnosine in muscles, thereby decreasing fatigue and increasing muscle work/output. A molecular weight of 89.09 g/mol ensures absorbability by nasal membranes.

Alternatively or in addition, Carnosine (B-alanyl-L-histidine) may be added to the solution. Carnosine supplementation helps keep the pH in the muscle balanced, has been shown to increase strength and endurance, speed up muscle recovery, and delay the onset of fatigue. A molar mass of 226.23 g/mol ensures nasal absorbability.

Alternatively or in addition, citrulline may be added to the solution. Citrulline is an essential amino acid that supports the optimization of blood flow to the tissues, thereby improving energy and speeding up recovery. Citrulline dramatically speeds up the rate at which the metabolic by-products of exercise (such as lactic acid and ammonia) are removed from the working muscle. Citrulline's molar mass of 175.19 g/mol ensures nasal absorbability, and citruline has little to no side effects.

Alternatively or in addition, creatine may be added to the composition in any known or future water-soluble form, such as creatine monohydrate, etc. Creatine is a natural nitrogenous organic acid that helps to supply energy to all cells in the body, primarily in skeletal muscles, thereby improving energy levels, endurance, and the ability to concentrate. Creatine's molar mass of 131.13 g/mol ensures nasal absorbability.

Alternatively or in addition, N-Dimethylglycine (DMG), an amino acid that is commonly known as glycine, may be added to the composition. Glycine is a metabolic enhancer, i.e. it improves oxygen utilization, and correspondingly, physical performance, while also inhibiting lactic acid. Glycine's molar mass of 103.12 g/mol ensures nasal absorbability. Moreover, Glycine is effective in small doses such as those administered via a nasal irrigation solution. Glycine is clear, water soluble, and odorless.

Alternatively or in addition, Isoleucine may be added to the solution in any known or future water-soluble form. Isoleucine is an essential amino acid, and is one of the branched chain amino acids (BCAAs) that increase protein synthesis in athletes during and after exercise, thereby resulting in increased stamina, and energy. Isoleucine's molar mass of 131.17 g/mol ensures nasal absorbability.

Alternatively or in addition, L-Phenylalanine LPA may be added to the solution in any known or future water-soluble form. Phenylalanine is an essential amino acid, and is a precursor for tyrosine, which promotes energy, mental focus, and motivation. Phenylalanine's molar mass of 165.19 g/mol ensures nasal absorbability.

Alternatively or in addition, L-Arginine may be added to the solution in any known or future water-soluble form. Arginine is a naturally-occurring non-essential amino acid that provides immune support, and is a precursor to Nitric oxide (NO), which enhances blood flow to working muscles, thereby improving oxygen and nutrient delivery and removal of waste products. Arginine's molar mass of 174.20 g/mol ensures nasal absorbability.

Alternatively or in addition, Leucine may be added to the solution in any known or future water-soluble form. Leucine is an essential amino acid (BCAA) that increases protein synthesis in athletes during and after exercise, thereby providing increased stamina and energy, and aids recovery. Leucine's molar mass of 131.17 g/mol ensures nasal absorbability.

Alternatively or in addition, N-Acetyl-Tyrosine (NAT) may be added to the solution in any known or future water-soluble form. NAT is a non-essential amino acid that is used to promote energy, mental focus, and motivation. NAT's molar mass of 181.19 g/mol ensures nasal absorbability. Moreover, NAT requires low doses to have a positive effect.

Alternatively or in addition, Phenylethylamine (PEA) may be added to the solution in any known or future water-soluble form. PEA is an essential amino acid that stimulates neurotransmitters in the brain that help with alertness. PEA's molar mass of 121.18 g/mol ensures nasal absorbability.

Alternatively or in addition, Taurine may be added to the solution in any known or future water-soluble form. Taurine is a naturally occurring sulfonic acid that may be added in conjunction with caffeine to balance the effects of caffeine. Taurine's molar mass of 125.15 g/mol ensures nasal absorbability.

Alternatively or in addition, Valine may be added to the composition in any known or future water-soluble form. Valine is an essential amino acid (one of the branched chain amino acids or BCAAs) that increases protein synthesis in athletes during and after exercise, provides increased stamina, increases muscle recovery, and boosts the immune system. Valine's molar mass of 117.15 g/mol ensures nasal absorbability.

In exemplary embodiments, one or more natural antioxidants or anti-inflammatory ingredients may be added to the salt and baking soda composition. These compounds may be added in addition to the alkaline earth metals and/or amino acids described above, and may be provided in a USP-grade form. The following compounds may be added by themselves or in any combination, resulting in a variety of compositions. For instance, Astaxanthin is an antioxidant that is naturally anti-inflammatory and provides potent antioxidant activity. Astaxanthin provides cardiovascular and immune benefits, and may be added to the composition in any known or future water-soluble form. Astaxanthin's molar mass of 596.84 g/mol ensures nasal absorbability, which is especially beneficial since Astaxanthin's absorption via other methods such as oral is limited.

Alternatively or in addition, coenzyme Q10 may be added to the composition in any known or future water-soluble form. Coenzyme Q10 is an antioxidant that functions in every cell of the body to synthesize energy. Coenzyme Q10's molar mass of 863.34 g/mol ensures nasal absorbability.

Alternatively or in addition, Ginseng may be added to the composition in any known or future water-soluble form. Ginseng is a natural plant based antioxidant that increases energy and stamina by enabling the muscles to use oxygen more effectively, and by regulating the metabolism and lowering recovery time. Ginseng's molar mass of 839.53 g/mol ensures nasal absorbability.

Alternatively or in addition, grape seed extract may be added to the composition in any known or future water-soluble form. Grape seed extract is a natural plant-based anti-inflammatory and antioxidant that combats oxidative stress and inflammation, particularly during physical exercise.

Alternatively or in addition, Quercetin may be added to the composition in any known or future water-soluble form. Quercetin is a plant-derived flavonoid and a natural anti-inflammatory that increases energy and endurance. Quercetin's molar mass of 302.236 g/mol ensures nasal absorbability.

Alternatively or in addition, Resveratrol may be added to the composition in any known or future water-soluble form. Resveratrol is a natural plant-based anti-oxidant and anti-inflammatory that improves energy and endurance, and lowers blood sugar levels. Resveratrol's molar mass of 228.24 g/mol ensures nasal absorbability.

Alternatively or in addition, Sodium Ascorbate (commonly known as Vitamin C) may be added to the composition in any known or future water-soluble form. Vitamin C is an antioxidant and anti-histamine that improves the health of the upper respiratory tract. Vitamin C's molar mass of 198.11 g/mol ensures nasal absorbability.

In exemplary embodiments, one or more natural plant-based stimulants may be added to the salt and baking soda composition. These ingredients may be added in addition to the alkaline earth metals, amino acids, and/or antioxidants described above, and may be provided in a USP-grade form. The following ingredients may be added by themselves or in any combination, resulting in a variety of compositions. For instance, caffeine may be added to the composition in any known or future water-soluble form. Caffeine is a central nervous system and metabolic stimulant that increases energy, endurance, and focus. Caffeine's molar mass of 194.19 g/mol ensures nasal absorbability. Caffeine is typically white when solid and colorless when dissolved.

Alternatively or in addition, Guarana may be added to the composition in any known or future water-soluble form. Guarana is a natural plant-based stimulant. Guarana seeds contain about twice the concentration of caffeine found in coffee beans. Guarana improves memory, alertness, and mood, thereby resulting in cognitive improvement.

Alternatively or in addition, synephrine may be added to the composition in any known or future water-soluble form. Synephrine is a natural plant-based stimulant that increases energy and has decongestant properties, i.e. beneficially constricting the nasal mucosa, thereby improving breathing performance, without irritating the membranes. Synephrine's molar mass of 167.21 g/mol ensures nasal absorbability.

Alternatively or in addition, Yerba mate may be added to the composition in any known or future water-soluble form. Yerba mate is a natural plant-based stimulant that is high in antioxidants and contains caffeine, thereby providing increased mental energy, clarity, and focus, and improving energy levels by helping the body use carbohydrates more efficiently. Yerba mate further balances out any potential negative effects of caffeine such as headaches, stomach irritation, jitters, etc.

In exemplary embodiments, one or more vitamins may be added to the salt and baking soda composition. These ingredients may be added in addition to the alkaline earth metals, amino acids, antioxidants, and/or stimulants described above, and may be provided in a USP-grade form. The following ingredients may be added by themselves or in any combination, resulting in a variety of compositions. For instance, methylcobalamin (known as vitamin B-12) may be added to the composition in any known or future water-soluble form. Vitamin B-12 increases focus, energy levels, and improves metabolic functions. Vitamin B-12's molar mass of 1344.40 g/mol ensures nasal absorbability.

Alternatively or in addition, vitamin D may be added to the composition in any known or future water-soluble form. Vitamin D maintains bone health and improves metabolic function. One or more forms of Vitamin D may be added, with Vitamin D2's molar mass of 396.65 g/mol and vitamin D3's molar mass of 384.64 g/mol ensuring nasal absorbability.

In exemplary embodiments, one or more synergists or other ingredients may be added to the salt and baking soda composition. These ingredients may be added in addition to the alkaline earth metals, amino acids, antioxidants, stimulants, and/or vitamins described above, and may be provided in a USP-grade form. The following ingredients may be added by themselves or in any combination, resulting in a variety of compositions. For instance, adenosine triphosphate (ATP) may be added to the composition in any known or future water-soluble form. ATP is a co-enzyme and it is the high-energy molecule that stores energy used by the muscles. Therefore, adding ATP to the composition may enhance energy, endurance, and overall performance. ATP's molar mass of 507.18 g/mol ensures nasal absorbability.

Alternatively or in addition, chromium picolinate may be added to the composition in any known or future water-soluble form. Chromium picolinate increases the efficiency of insulin in the body to optimal levels, thereby promoting energy, and increasing strength and lean muscle mass. Chromium picolinate's molar mass of 418.33 g/mol ensures nasal absorbability.

Alternatively or in addition, medium-chain triglycerides (MCTs) may be added to the composition in any known or future form, such as MCT powder, or coconut oil. MCTs improve absorption of baking soda, thereby acting as a synergist. This increases energy levels.

Alternatively or in addition, colostrum may be added to the composition in any known or future water-soluble form. Colostrum is a type of milk produced by the mammary glands of mammals in late pregnancy. Colostrum boosts the immune system, reduces risk of UR illness, and strengthens the stomach wall, which is especially beneficial for athletes who develop gut problems/cramping during intense periods of training. Only a small amount (approximately two grams) is required for a beneficial effect.

Alternatively or in addition, glucose may be added to the composition in any known or future water-soluble form. Glucose is a simple monosaccharide that is found in plants. Glucose is the human body's key source of energy, and a primary source of energy for the brain. Glucose's molar mass of 180.16 g/mol ensures nasal absorbability. Glucose is typically colorless and easily soluble in water.

Alternatively or in addition, menthol may be added to the composition in any known or future water-soluble form. Menthol acts as a breathing aid and decongestant, thereby improving performance. Menthol also blocks voltage-sensitive sodium channels, reducing neural activity that may stimulate muscles. Menthol has the added benefit of providing a palatable taste, and menthol's molar mass of 156.27 g/mol ensures nasal absorbability.

Alternatively or in addition, ribose may be added to the composition in any known or future water-soluble form. Ribose is a natural monosaccharide that boosts muscle recovery and improves muscular stamina. Further, Ribose's molar mass of 150.13 g/mol ensures nasal absorbability.

Some of the ingredients disclosed above may not be available in a water-soluble form. For such ingredients, a grain or mesh size may be selected such that mixing them in water along with the salt and baking soda (and any other ingredient) may result in a colloidal or emulsified version of the ingredient. The solution would then serve to deliver the insoluble particles across the nasal membranes for optimal absorption.

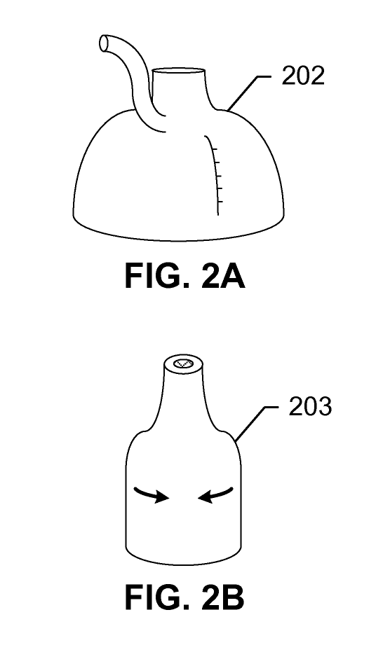

FIGS. 2A and 2B show containers for nasal irrigation, according to exemplary embodiments of the subject disclosure. FIG. 2A shows a container 202 that includes a spout for insertion into a nostril, wherein container 202 is tilted at an angle allowing the resulting solution to flow into the nostril and is released from the second nostril by action of gravity, similar to existing neti-pot systems. Container 202 may depict a scale for measuring a fixed volume of water, and may be included in a nasal irrigation kit along with one or more powder packets containing compositions such as those described with respect to FIG. 1. FIG. 2B depicts a squeezable container 203 that is intended to spray a nasal irrigation solution into a nostril when squeezed. This is an alternative container to that of FIG. 2A, and may be pre-mixed and ready to use upon breaking a seal on top of the container, or may be prepared by a user. One or both of containers 202 and 203 may be included in a kit purchased by a first-time user, with an option to purchase additional powder packets of different compositions if needed.

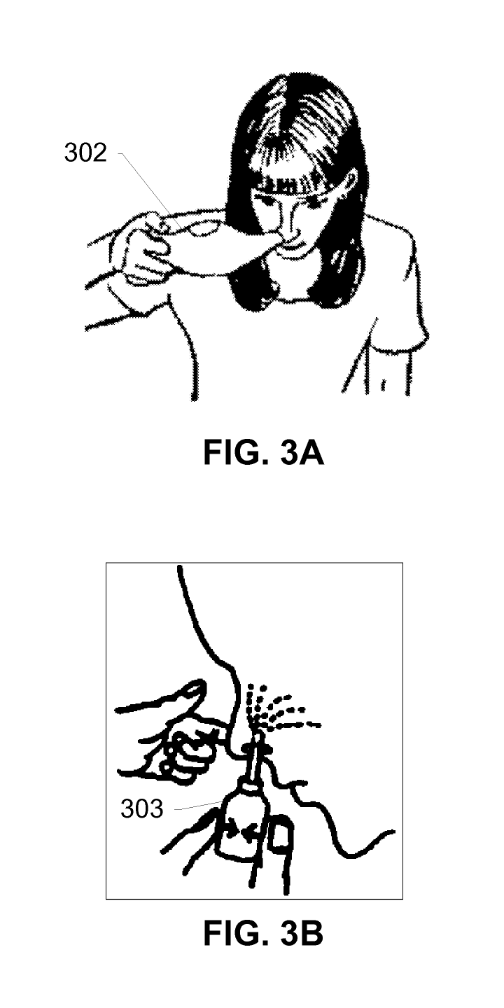

FIGS. 3A and 3B show methods for nasal irrigation, according to exemplary embodiments of the subject disclosure. FIG. 3A shows a user inserting a spout of container 302 into a nostril, and tilting container 302 at an angle, allowing the resulting solution to flow from the first nostril and out of the second nostril by action of gravity, similar to existing neti-pot systems. FIG. 3B depicts a squeezable container 303 that is intended to spray a nasal irrigation solution into a nostril when squeezed. The composition in container 303 may be pre-mixed and ready to use upon breaking a seal on top of the container, or may be prepared by a user.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A composition for nasal irrigation, comprising:
a first quantity of anhydrous sodium chloride sufficient to form a fixed volume of a solution having a salinity equivalent to a nasal mucosa; and
a second quantity of sodium bicarbonate sufficient to neutralize a pH of the solution to match a pH of the nasal mucosa.

2. The composition of claim 1, further comprising a third quantity of an alkaline earth metal.

3. The composition of claim 2, wherein the alkaline earth metal is magnesium.

4. The composition of claim 3, wherein the magnesium is in the form of magnesium malate.

5. The composition of claim 1, further comprising one or more amino acids.

6. The composition of claim 5, wherein the one or more amino acids includes citrulline.

7. The composition of claim 5, wherein the one or more amino acids includes glycine.

8. The composition of claim 1, further comprising one or more natural antioxidants.

9. The composition of claim 1, further comprising one or more plant-based stimulants.

10. The composition of claim 9, wherein the one or more plant-based stimulants includes caffeine.

11. The composition of claim 1, further comprising one or more vitamins.

12. The composition of claim 1, further comprising menthol.

13. The composition of claim 1, further comprising glucose.

14. An apparatus for nasal irrigation, comprising:
a container for carrying a fixed volume of water; and
an anhydrous composition comprising a first quantity of sodium chloride and a second quantity of sodium bicarbonate;
wherein a solution of the first quantity of sodium chloride and the second quantity of sodium bicarbonate in the fixed volume of water has a salinity and a pH equivalent to that of a nasal mucosa.

15. The apparatus of claim 14, wherein the anhydrous composition further comprises magnesium malate.

16. The apparatus of claim 14, wherein the anhydrous composition further comprises caffeine.

17. The apparatus of claim 14, wherein the anhydrous composition is pre-mixed with the water to form the solution.

18. A method for enhancing performance, comprising:
irrigating a nasal membrane using a fixed volume of a solution comprising a first quantity of sodium chloride and a second quantity of sodium bicarbonate;
wherein the solution has a salinity and a pH equivalent to that of a nasal mucosa.

19. The method of claim 18, further comprising providing an anhydrous powder packet comprising the first quantity of sodium chloride and the second quantity of sodium bicarbonate, wherein the powder packet is mixed with the fixed volume of water to form the fixed volume of the solution.

20. The method of claim 18, wherein the anhydrous composition further comprises one or more of magnesium malate, caffeine, menthol, or glucose.
